(19) United States
(12) Patent Application Publication
(10) Pub. No.: US 2011/0123459 A1
(43) Pub. Date: May 26, 2011

(54) Anaerobic Iodine Solution

(76) Inventors: William Scott Whiting, The Woodlands, TX (US); John E. Stalcup, The Woodlands, TX (US)

(21) Appl. No.: 12/625,325

(22) Filed: Nov. 24, 2009

(51) Int. Cl. A61K 33/18; A61K 9/12; A61P 31/04; B01D 47/02

(52) U.S. Cl. 424/43; 424/667; 424/668; 424/670; 261/121.1

(57) ABSTRACT

In accordance with the present invention, iodine, an iodine derivative(s), or a combination of a plurality of these substances is presented as the active ingredient of a stable naturopathic liquid solution. More specifically, in addition to the active ingredient, the naturopathic solution will include a liquid carrier that has been saturated with an inert gas (e.g. Nitrogen) to improve the stability of the active ingredient and increase the shelf life of the solution. For the manufacture of the solution, a liquid carrier (e.g. purified distilled water) is degassed and then subsequently saturated with the inert gas. The active ingredient is then added to the saturated liquid carrier to create the naturopathic liquid solution.

### FIELD OF THE INVENTION

The present invention pertains generally to naturopathic solutions that can be vaporized and inhaled, or topically applied, to treat a variety of infections. More particularly, the present invention pertains to naturopathic solutions that incorporate iodine and/or an iodine derivative(s) as the active ingredient(s). The present invention is particularly, but not exclusively, useful as an iodine solution that has been degassed (e.g. deoxygenated) and subsequently saturated with an inert gas to stabilize the iodine in solution for improved shelf-life.

### BACKGROUND OF THE INVENTION

Iodine is a nonmetallic chemical element belonging to the halogen group. It is well known, inter alia, for its antiseptic and curative qualities. Indeed, it is known there are many beneficial applications for properly concentrated iodine. Iodine, however, is very reactive, and is particularly unstable when placed in solution. Specifically, it is known that when exposed to either oxygen, single cell microbes, metal containers, some plastics or other impurities, a simple iodine solution will quickly loose its efficacy. Although the addition of other chemicals into an iodine solution can improve its overall stability, this may not be a commercially viable alternative. Instead, a naturopathic solution is preferred.

For a naturopathic iodine solution, an important consideration is that there be a minimal use of ingredients that are reactive with iodine. With this in mind, when a naturopathic iodine solution is to be manufactured, it is important there be nothing included that will react with iodine and/or its derivatives. With this in mind, a variety of carriers may be used to enhance the effects of the solution even though they might be considered active for treating an ailment. For example, sugar alcohols such as glycerin or xylitol may have beneficial qualities as a wetting agent but will have minimal, if any, reaction with iodine. Consequently, factors that contribute to the instability of an iodine solution become important. Of particular interest here is the fact the iodine instability may actually involve the liquid carrier that is used to create the iodine solution. For instance, an otherwise suitable liquid carrier for an iodine solution is water. Water, however, includes oxygen which is known to adversely affect the stability of iodine.

In light of the above, it is an object of the present invention to provide a stable (e.g. anaerobic) naturopathic iodine solution having an increased shelf life. Another object of the present invention is to provide a stable naturopathic iodine solution that can have varied concentrations of iodine, and be either vaporized for inhalation or used directly and topically applied, to treat a variety of infections. Still another object of the present invention is to provide an anaerobic naturopathic iodine solution that is easy to manufacture, is simple to use and is comparatively cost effective.

### SUMMARY OF THE INVENTION

For the present invention, a stable (e.g. anaerobic) naturopathic iodine solution and a system for manufacturing the solution in accordance with a particular methodology are disclosed. Specifically, the solution is prepared (i.e. manufactured) in a manner that improves the stability of the iodine solution, to thereby increase its shelf life. Further, the present invention envisions an iodine solution that can be used as a liquid or a liquid vapor for the treatment of a plethora of afflictions.

With the above in mind, an anaerobic naturopathic liquid solution in accordance with the present invention includes an active ingredient (i.e. substance) that is either iodine, an iodine derivative(s), or various combinations of these substances. Preferably, the iodine derivative is potassium iodide, potassium iodate or povidone iodine. In addition to the active ingredient, the solution also includes an inert gas (e.g. Nitrogen) and a degassed liquid carrier (e.g. distilled water, saline solution, xylitol, glycerin or various combinations of these carriers). Importantly, the liquid carrier is substantially saturated with the inert gas. For the present invention, the active ingredient (substance) and the inert gas are provided in solution with the degassed carrier.

In accordance with the present invention, a system for preparing the anaerobic naturopathic solution includes a container that is formed with a chamber for holding liquids. For purposes of the system of the present invention, the container will function alternatively as a pressure chamber and as a vacuum chamber. Preferably, the container is made of glass. The container may, however, be made of a metal that is lined with an inert protective barrier (e.g. Teflon®). Importantly, the system also includes a bubbler tube that is positioned inside the chamber to essentially extend through the chamber. Specifically, the bubbler tube has a first end that is affixed to the wall of the container, and it has a second end that is positioned in the chamber, across the chamber from the first end.

A vacuum pump is connected in fluid communication with the chamber to selectively create a partial vacuum inside the chamber. There is also a source of a pressurized inert gas (e.g. Nitrogen) that is connected in fluid communication with the first end of the bubbler tube, and there is a source of the liquid carrier that will be used to fill the chamber. Additionally, there is a source of the substance (i.e. iodine and/or an iodine derivative) that will be introduced into the chamber through an access port, after the liquid carrier has been degassed and saturated with the inert gas.

Control for the overall operation of the system can be accomplished by a computer that is electronically connected with the source of pressurized inert gas and with the vacuum pump, The purpose here is to concertedly control both the source of pressurized inert gas and the vacuum pump for preparation of the anaerobic naturopathic solution in accordance with a predetermined procedure. The computer can also be connected with an agitator for appropriately vibrating the container to agitate the solution in the chamber after the active ingredient has been added to the saturated carrier.

As for the predetermined procedure mentioned above, the present invention includes a method for preparing an anaerobic naturopathic solution that requires the container first be purged with the inert gas. The container can then be filled with the liquid carrier, and a vacuum can be created in the container by the vacuum pump, to degas the liquid carrier. Once the liquid carrier has been degassed, the inert gas is bubbled through the degassed carrier to reestablish an ambient pressure in the container. As envisioned for the present invention, cycles of the liquid carrier being degassed, followed by the inert gas being bubbled through the degassed carrier will be repeated at least three times. After the cycles have been completed, the container will be pressurized above ambient pressure with the inert gas to further saturate the degassed carrier with the inert gas. When the carrier is saturated with the inert gas, and ambient pressure has been reestablished inside the container, the active ingredient (i.e. substance) can be introduced into the container to create the anaerobic naturopathic solution.

Additional steps in the method of the present invention, after the anaerobic naturopathic solution has been created, may include another over-pressurizing of the container with the inert gas to ensure saturation. And, the container can be agitated to mix the anaerobic naturopathic solution. Further, after the agitating step, a vacuum may again be drawn in the container to further degas the anaerobic naturopathic solution, and there may then be additional bubbling of the inert gas through the anaerobic naturopathic solution to increase the concentration of inert gas in the solution and to reestablish ambient pressure in the container. This must then be followed by a period of pressurization with an inert gas to ensure the solution will be saturated and to help off-gassing during the bottling process. The solution should remain pressurized until it is released into the bottles and immediately capped.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic representation of components for the system of the present invention; and

FIG. 2 shows a sequence of steps for performing a procedure in accordance with the present invention.

### DESCRIPTION OF THE PREFERRED EMBODIMENTS

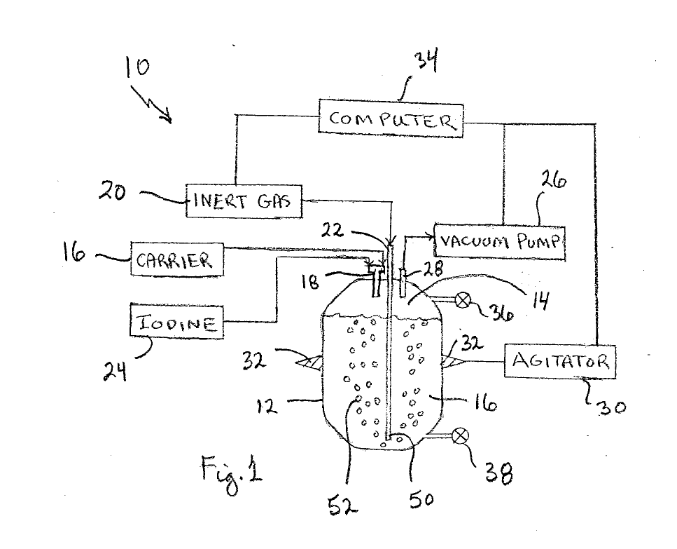

Referring initially to FIG. 1, a system for preparing an anaerobic naturopathic liquid solution in accordance with the present invention is shown and is generally designated 10. In FIG. 1 it will be seen that the system 10 includes a container 12 which is formed with a chamber 14. For the system 10, the container 12 is preferably made of glass; however, it can be made of metal if it is lined with an inert material (e.g. Teflon®). Also, a source of a liquid carrier 16 is provided, and this source is connected in fluid communication with an access port 18 that allows the introduction of the liquid carrier 16 into the chamber 14 of container 12. The system 10 also includes a source of a pressurized inert gas 20 that can be introduced into the chamber 14 via a bubbler tube 22. As envisioned for the present invention, the inert gas 20 is preferably Nitrogen, and the liquid carrier 16 is preferably selected from a group consisting of purified distilled water, saline solution, xylitol, glycerin or various combinations of these carriers. FIG. 1 also shows that the system 10 includes a source of an active ingredient 24 that can selectively be introduced into the chamber 14 through the access port 18. As indicated in FIG. 1 for the present invention, the active ingredient 24 is either iodine or an iodine derivative. Preferably, the iodine derivative may be potassium iodide, potassium iodate or povidone iodine. Importantly, rather than being either pure iodine or an iodine derivative, the active ingredient 24 can also be a combination of these elements.

Still referring to FIG. 1, the system 10 is shown to include a vacuum pump 26 that is connected in fluid communication with the chamber 14 via a valve 28. Additionally, a mechanical agitator 30 is connected to the container 12 via a grip 32. Further, a computer 34 can be employed to control the system 10 as generally indicated in FIG. 1. Specifically, electronic connections can be established between the computer 34 and the source of inert gas 20, the vacuum pump 26 and the agitator 30 to coordinate their various functions. Additionally, the source of liquid carrier 16 and the source of the active ingredient 24 can be controlled by the computer 34, if desired. As will be best appreciated from the discussion below regarding an operation of system 10, a pressure relief valve 36 and an output port 38 can be included as part of the container 12.

### Operation

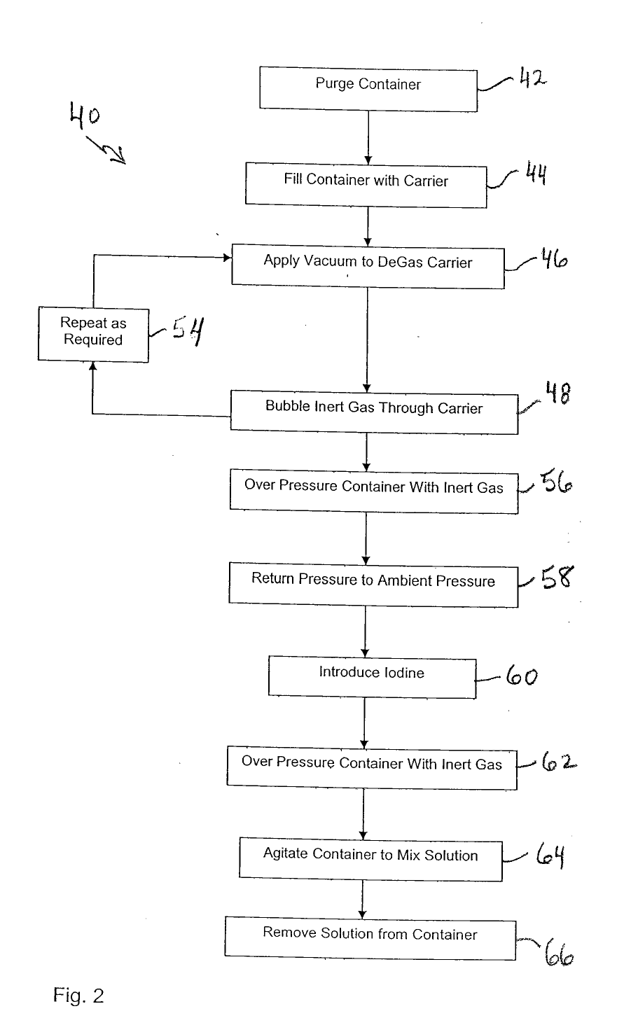

With reference to FIG. 2, an operation of the system 10 is outlined by the function chart shown in FIG. 2 which is generally designated 40. As a first step in the operation of the system 10, block 42 of chart 40 indicates that the chamber 14 of container 12 is purged by the inert gas 20. Though not shown, it is to be appreciated that during this purging step, the chamber 14 is empty. Next, as indicated by block 44 in the chart 40, after the chamber 14 has been purged, it is partially filled with the liquid carrier 16 (e.g. preferably purified distilled water). A vacuum is then created in the chamber 14 by the vacuum pump 26 to degas (deoxygenate) the liquid carrier 16 (block 46). This vacuum can be continued while the inert gas 20 is passed through the bubbler tube 22 as indicated by block 48 in the chart 40. Specifically, as the inert gas 20 leaves the end 50 of bubbler tube 22, it forms gas bubbles 52 in the liquid carrier 16 that contribute to the solution of the inert gas 20 in the liquid carrier 16. The actions indicated by blocks 46 and 48 can then be repeated, if desired, as indicated by block 54. This is done for the purpose of saturating the liquid carrier 16 with the inert gas 20. To further ensure saturation of inert gas 20 in the liquid carrier 16, block 56 indicates that the chamber 14 is subjected to an overpressure of the inert gas 20. After an appropriate period of time, the pressure relief valve 36 can then be activated to return the chamber 14 to ambient pressure (see block 58).

Once the liquid carrier 16 has been substantially saturated with the inert gas 20, block 60 indicates the active ingredient (i.e. iodine) 24 is introduced into the chamber 14. This is done through the access port 18 in order to mix the active ingredient 24 with the now-saturated liquid carrier 16. Again, the chamber 14 of container 12 can be subjected to an overpressure of the inert gas 20 (see block 62) to further insure saturation of the liquid carrier 16. As a final step in the process, the agitator 30 can be activated to agitate the container 12 with grip 32 to mix the solution of inert gas 20 and liquid carrier 16 (block 64). This solution can then be removed from the container 12 via the output port 38 and bottled, or otherwise packaged, for commercial consumption (block 66). It is an important concept of the present invention that all steps for the process shown in function chart 40 can be initiated and subsequently controlled for specified time durations in accordance with instructions from computer 34.

While the particular Anaerobic Iodine Solution as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A stable naturopathic liquid solution comprising:
a substance selected from a group including iodine and an iodine derivative;
an inert gas; and
a degassed liquid carrier, wherein the degassed liquid carrier has been substantially saturated with the inert gas and wherein the substance is provided in solution with the saturated carrier to create the solution.

2. A solution as recited in claim 1 wherein the iodine derivative is selected from a group including potassium iodide, potassium iodate, and povidone iodine, and the substance is a combination of a plurality of selected substances.

3. A solution as recited in claim 1 wherein the inert gas is Nitrogen.

4. A solution as recited in claim 1 wherein the degassed carrier is selected from a group including purified distilled water, saline solution, xylitol, glycerin, and combinations thereof.

5. A solution as recited in claim 1 wherein the inert gas is Nitrogen and the degassed carrier is purified distilled water.

6. A solution as recited in claim 1 wherein the solution is anaerobic.

7. A solution as recited in claim 1 wherein the solution is provided as a spray.

8. A method for preparing a stable naturopathic solution which comprises the steps of:
providing a container;
purging the container with an inert gas;
filling the container with a liquid carrier;
applying a vacuum in the container to degas the liquid carrier;
bubbling the inert gas through the degassed carrier to reestablish an ambient pressure in the container;
sequentially repeating the applying step and the bubbling step, as required;
pressurizing the container above ambient pressure with the inert gas to saturate the degassed carrier with the inert gas;
reestablishing ambient pressure in the container; and
introducing a substance into the container to create the naturopathic solution, wherein the substance is selected from a group including iodine and an iodine derivative.

9. A method as recited in claim 8 further comprising the steps of:
over-pressurizing the container with the inert gas; and
agitating the container to mix the anaerobic naturopathic solution.

10. A method as recited in claim 9 further comprising, after the agitating step, the steps of:
applying a vacuum in the container to further degas the anaerobic naturopathic solution;
bubbling the inert gas through the naturopathic solution to reestablish ambient pressure in the container; and
pressurizing the container with the inert gas.

11. A method as recited in claim 8 wherein the container is made of glass.

12. A method as recited in claim 9 wherein the iodine derivative is selected from a group consisting of potassium iodide, potassium iodate, and povidone iodine, and the substance is a combination of a plurality of selected substances.

13. A method as recited in claim 9 wherein the inert gas is Nitrogen.

14. A method as recited in claim 9 wherein the degassed carrier is selected from a group including purified distilled water, saline solution, xylitol, glycerin and combinations thereof.

15. A method as recited in claim 9 wherein the sequentially repeating step is accomplished at least three times.

16. A system for preparing a stable naturopathic solution which comprises:
a container for holding liquids, with the chamber being bounded by a wall formed with an access port;
a bubbler tube positioned inside the chamber, the bubbler tube having a first end and a second end, with the first end thereof affixed to the wall of the container, and with the second end positioned in the chamber across the chamber from the first end;
a source of a pressurized inert gas connected in fluid communication with the first end of the bubbler tube;
a vacuum pump connected in fluid communication with the chamber for selectively creating a partial vacuum inside the chamber;
a source of a liquid carrier for selective introduction of the liquid carrier into the chamber through the access port;
a source of a substance for selective introduction of the substance into the chamber through the access port;
a computer electronically connected with the source of pressurized inert gas and with the vacuum pump to concertedly control the source of pressurized inert gas and the vacuum pump for operation of the system in accordance with a predetermined procedure; and
an agitator for vibrating the container to agitate the solution in the chamber.

17. A system as recited in claim 16 wherein the inert gas is Nitrogen and the liquid carrier is distilled water.

18. A system as recited in claim 16 further comprising a pressure relief valve mounted on the container for selectively removing gas from the chamber, as required.

19. A system as recited in claim 16 further comprising a drain valve mounted on the container for removing the naturopathic solution from the chamber.

20. A system as recited in claim 16 wherein the container is made of glass.
